(19) United States
(12) Patent Application Publication
(10) Pub. No.: US 2012/0059241 A1
(43) Pub. Date: Mar. 8, 2012

(54) SYSTEMS AND METHODS FOR MAKING AND USING A STEERABLE IMAGING SYSTEM CONFIGURED AND ARRANGED FOR INSERTION INTO A PATIENT

(75) Inventors: Roger Hastings, Maple Grove, MN (US); Tat-Jin Teo, Sunnyvale, CA (US); Frank W. Ingle, Palo Alto, CA (US); Kevin D. Edmunds, Ham Lake, MN (US)

(73) Assignee: BOSTON SCIENTIFIC SCIMED, INC., Maple Grove, MN (US)

(21) Appl. No.: 13/225,962

(22) Filed: Sep. 6, 2011

Related U.S. Application Data
(60) Provisional application No. 61/380,951, filed on Sep. 8, 2010.

(51) Int. Cl. A61B 5/055; A61B 8/12

(52) U.S. Cl. 600/409; 600/462

(57) ABSTRACT

A medical imaging assembly includes a sheath with a lumen. An imaging core is disposed at one end of an imaging core shaft disposed in the lumen. The imaging core shaft bends along a shape memory region when the imaging core is extended from the lumen. The imaging core includes a transducer to image patient tissue, a mirror to redirect acoustic signals between the transducer and patient tissue, and a magnet to drive rotation of the mirror. The magnet is rotatable by a magnetic field generated at the location of the magnet. An imaging core shaft rotator rotates the imaging core shaft such that, when the imaging core is extended from the lumen, rotation of the imaging core shaft causes radial rotation of the imaging core about the sheath. The imaging core shaft rotator includes rotatable imaging core shaft magnets fixedly disposed over a portion of the imaging core shaft.

### CROSS REFERENCE TO RELATED APPLICATIONS

This application claims the benefit of U.S. Provisional Application No. 61/380,951, entitled, “SYSTEMS AND METHODS FOR MAKING AND USING A STEERABLE IMAGING SYSTEM CONFIGURED AND ARRANGED FOR INSERTION INTO A PATIENT,” by Roger Hastings, Tat-Jin Teo, Frank W. Ingle, and Kevin D. Edmunds, and filed on Sep. 8, 2010, the entire contents of which being incorporated herein by reference.

### TECHNICAL FIELD

The present invention is directed to the area of imaging systems that are insertable into a patient and methods of making and using the imaging systems. The present invention is also directed to imaging systems with imaging cores that can be steered into various angular orientations within a patient using magnetic motors, as well as methods of making and using the imaging systems, imaging cores, and magnetic motors.

### BACKGROUND

Ultrasound devices insertable into patients have proven diagnostic capabilities for a variety of diseases and disorders. For example, intravascular ultrasound (“IVUS”) imaging systems have been used as an imaging modality for diagnosing blocked blood vessels and providing information to aid medical practitioners in selecting and placing stents and other devices to restore or increase blood flow. IVUS imaging systems have been used to diagnose atheromatous plaque build-up at particular locations within blood vessels. IVUS imaging systems can be used to determine the existence of an intravascular obstruction or stenosis, as well as the nature and degree of the obstruction or stenosis. IVUS imaging systems can be used to visualize segments of a vascular system that may be difficult to visualize using other intravascular imaging techniques, such as angiography, due to, for example, movement (e.g., a beating heart) or obstruction by one or more structures (e.g., one or more blood vessels not desired to be imaged). IVUS imaging systems can be used to monitor or assess ongoing intravascular treatments, such as angiography and stent placement in real (or almost real) time. Moreover, IVUS imaging systems can be used to monitor one or more heart chambers.

IVUS imaging systems have been developed to provide a diagnostic tool for visualizing a variety is diseases or disorders. An IVUS imaging system can include a control module (with a pulse generator, an image processor, and a monitor), a catheter, and one or more transducers disposed in the catheter. The transducer-containing catheter can be positioned in a lumen or cavity within, or in proximity to, a region to be imaged, such as a blood vessel wall or patient tissue in proximity to a blood vessel wall. The pulse generator in the control module generates electrical pulses that are delivered to the one or more transducers and transformed to acoustic pulses that are transmitted through patient tissue. Reflected pulses of the transmitted acoustic pulses are absorbed by the one or more transducers and transformed to electric pulses. The transformed electric pulses are delivered to the image processor and converted to an image displayable on the monitor.

Intracardiac echocardiography (“ICE”) is another ultrasound imaging technique with proven capabilities for use in diagnosing intravascular diseases and disorders. ICE uses acoustic signals to image patient tissue. Acoustic signals emitted from an ICE imager disposed in a catheter are reflected from patient tissue and collected and processed by a coupled ICE control module to form an image. ICE imaging systems can be used to image tissue within a heart chamber.

### BRIEF SUMMARY

In one embodiment, a medical imaging assembly includes an elongated sheath having a proximal end, an open distal end, and a longitudinal axis. The sheath defines a lumen that extends to the open distal end. An imaging core shaft is at least partially disposed in the lumen. The imaging core shaft has a proximal end, a distal end, and a longitudinal axis. The distal end of the imaging core shaft includes a shape memory region. A sealed imaging core is disposed at the distal end of the imaging core shaft. The imaging core is configured and arranged for extending outward from the open distal end of the sheath. When the shape memory region of the imaging core shaft is at least partially extended from the distal end of the sheath, the shape memory region is configured and arranged to bend axially with respect to the longitudinal axis of the sheath such that the longitudinal axis of the sheath is not parallel with the longitudinal axis of the imaging core. The imaging core includes an imaging core magnet disposed at a location in the imaging core. The imaging core magnet is configured and arranged to rotate at a target frequency by generation of a magnetic field at the location of the imaging core magnet. At least one transducer is configured and arranged for transforming applied electrical signals to acoustic signals, transmitting the acoustic signals, receiving corresponding echo signals, and transforming the received echo signals to electrical signals. The imaging core also includes a mirror with a reflective surface configured and arranged for reflecting acoustic signals transmitted from the at least one transducer and corresponding echo signals. The mirror is coupled to the imaging core magnet such that rotation of the imaging core magnet causes a corresponding rotation of the mirror. An imaging core shaft rotator is configured and arranged to rotate the imaging core shaft such that, when the imaging core is at least partially extended from the open distal end of the sheath, rotation of the imaging core shaft causes a corresponding radial rotation of the imaging core about the longitudinal axis of the sheath. The imaging core shaft rotator includes a plurality of rotatable imaging core shaft magnets fixedly disposed over a portion of the imaging core shaft. At least one transducer conductor is electrically coupled to the at least one transducer and is in electrical communication with the proximal end of the catheter.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an ultrasound imaging system suitable for insertion into a patient, according to the invention;

FIG. 2 is a schematic side view of one embodiment of a catheter suitable for use with the ultrasound imaging system of FIG. 1, according to the invention;

FIG. 3 is a schematic longitudinal cross-sectional view of one embodiment of a distal end of the catheter of FIG. 2 with an imaging core disposed in a lumen defined in a sheath, according to the invention;

FIG. 4 is a schematic perspective view of one embodiment of a mirror holder suitable for use with the imaging core of FIG. 3, according to the invention;

FIG. 5 is a schematic longitudinal cross-sectional view of one embodiment of imaging core shaft magnets and spacers disposed around a portion of the imaging core shaft of FIG. 3, the imaging core shaft disposed in the sheath of FIG. 3, according to the invention;

FIG. 6 is a schematic perspective view of one embodiment of magnetic-field windings embedded in walls of the sheath of FIG. 3, the magnetic-field windings suitable for driving the imaging core shaft magnets of FIG. 5 to steer the imaging core of FIG. 3, according to the invention;

FIG. 7 is a schematic view of one embodiment of the imaging core of FIG. 3 extended from a distal end of the sheath of FIG. 3, the imaging core tethered to the sheath by the imaging core shaft, the imaging core shaft including an axially bent distal region external to the sheath, according to the invention;

FIG. 8 is a schematic view of one embodiment of the imaging core of FIG. 3 extended from a distal end of the sheath of FIG. 3, the distal end of the sheath being axially bent, according to the invention; and

FIG. 9 is a schematic transverse cross-sectional view of one embodiment of one of the imaging core shaft magnets of FIG. 5 disposed in the sheath of FIG. 3, according to the invention.

### DETAILED DESCRIPTION

The present invention is directed to the area of imaging systems that are insertable into a patient and methods of making and using the imaging systems. The present invention is also directed to imaging systems with imaging cores that can be steered into various angular orientations within a patient using magnetic motors, as well as methods of making and using the imaging systems, imaging cores, and magnetic motors.

Suitable intravascular ultrasound (“IVUS”) and intracardiac echocardiography (“ICE”) systems include, but are not limited to, one or more transducers disposed on a distal end of a catheter configured and arranged for percutaneous insertion into a patient. Examples of IVUS imaging systems with catheters are found in, for example, U.S. Pat. Nos. 7,246,959; 7,306,561; and 6,945,938; as well as U.S. Patent Application Publication Nos. 2006/0100522; 2006/0106320; 2006/0173350; 2006/0253028; 2007/0016054; and 2007/0038111; all of which are incorporated herein by reference.

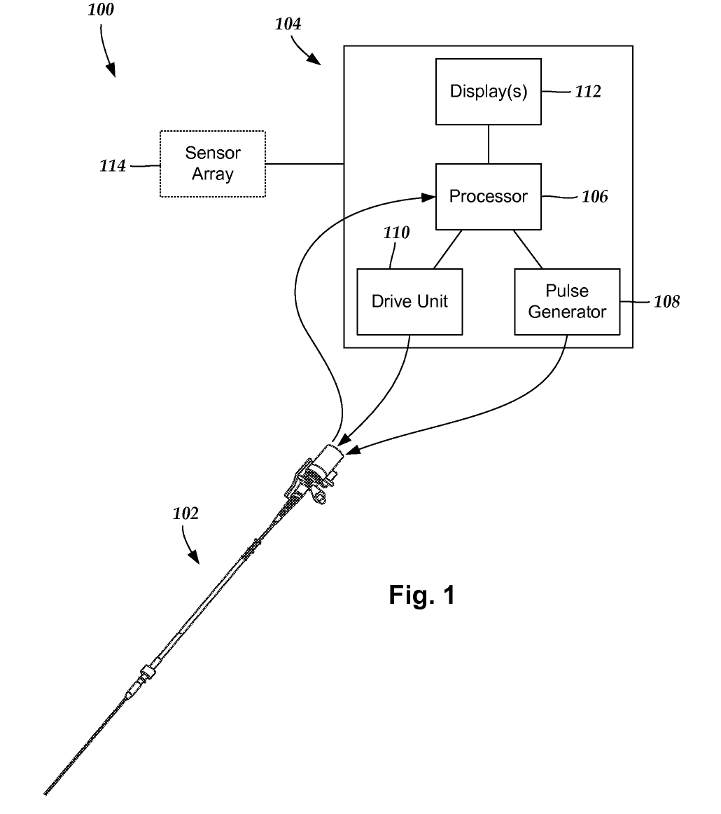

FIG. 1 illustrates schematically one embodiment of an IVUS imaging system 100. An ICE imaging system is similar. The IVUS imaging system 100 includes a catheter 102 that is coupleable to a control module 104. The control module 104 may include, for example, a processor 106, a pulse generator 108, a drive unit 110, and one or more displays 112. In at least some embodiments, the IVUS imaging system 100 further includes a sensor array 114 configured and arranged for detecting one or more magnetic fields within a patient. In at least some embodiments, the sensor array 114 determines the position, the orientation, or both of the one or more magnetic fields within the patient.

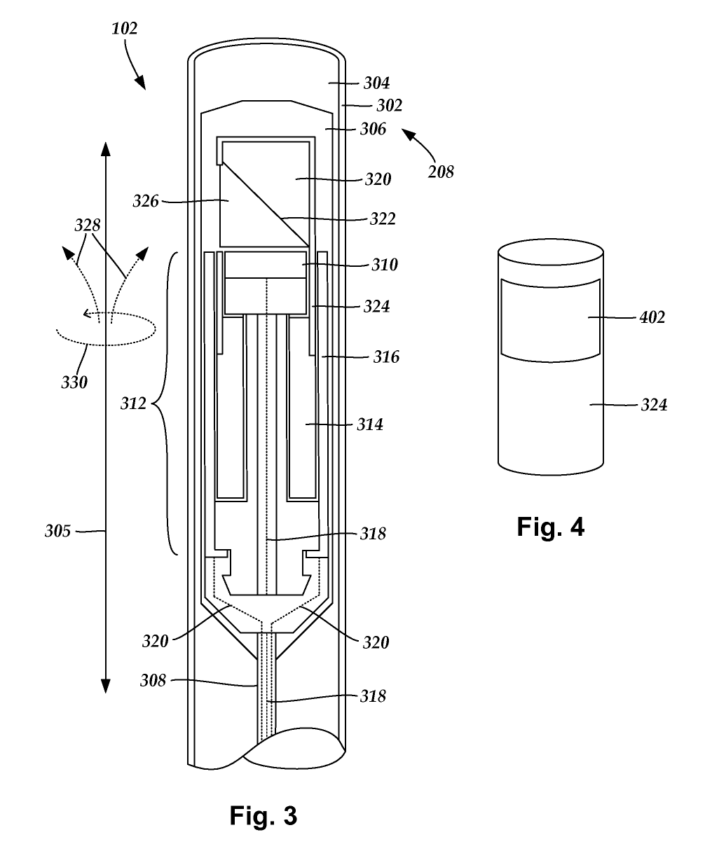

In at least some embodiments, the pulse generator 108 forms electric pulses that may be input to an imaging device (310 in FIG. 3), such as one or more ultrasound transducers, disposed in the catheter 102. In at least some embodiments, mechanical energy from the drive unit 110 may be used to drive pullback of an imaging core (306 in FIG. 3) disposed in the catheter 102. In at least some embodiments, pullback of the imaging core (306 in FIG. 3) can be performed manually in lieu of using mechanical energy from the drive unit 110. In at least some embodiments, the imaging core (306 in FIG. 3) can be pulled back through a sheath (302 in FIG. 3) (or over a guide wire) during an imaging procedure. This would enable the formation of 3-D reconstructions at differing levels along a blood vessel, or in a heart chamber.

In at least some embodiments, electric pulses transmitted from the imaging device (310 in FIG. 3) may be input to the processor 106 for processing. In at least some embodiments, the processed electric pulses from the imaging device (310 in FIG. 3) may be displayed as one or more images on the one or more displays 112. In at least some embodiments, the processor 106 may also be used to control the functioning of one or more of the other components of the control module 104. For example, the processor 106 may be used to control at least one of the frequency or duration of the electrical pulses transmitted from the pulse generator 108, the velocity or length of the pullback of the imaging core (306 in FIG. 3) by the drive unit 110, or one or more properties of one or more images formed on the one or more displays 112. In some embodiments, the parts of the control module 104 (i.e., the processor 106, the pulse generator 108, the drive unit 110, the one or more displays 112, and the sensor array 114) may be in one unit. In other embodiments, the parts of the control module 104 are in two or more units.

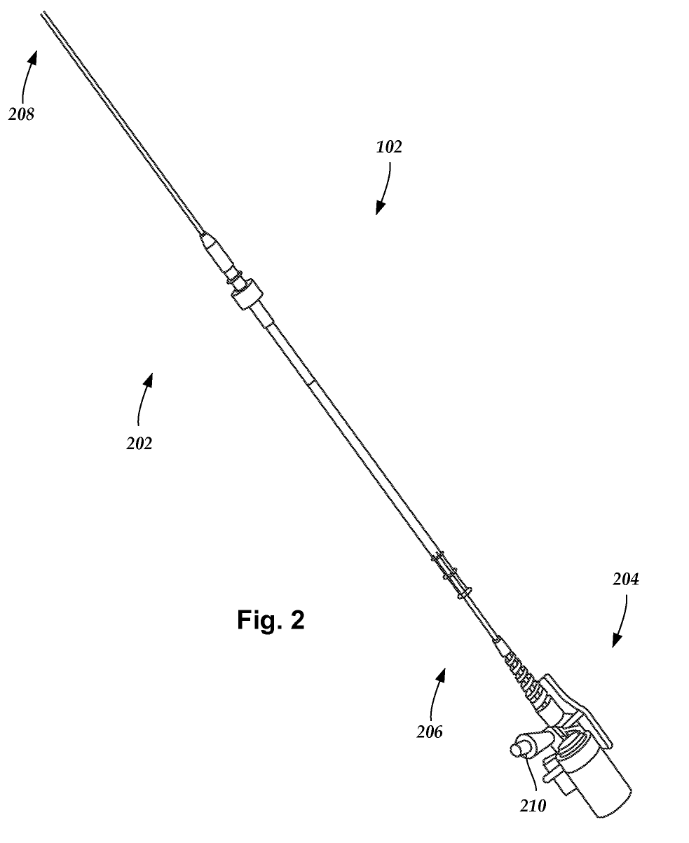

FIG. 2 is a schematic side view of one embodiment of the catheter 102 of the IVUS imaging system (100 in FIG. 1). The catheter 102 includes an elongated member 202 and a hub 204. The elongated member 202 includes a proximal end 206 and a distal end 208. In FIG. 2, the proximal end 206 of the elongated member 202 is coupled to the catheter hub 204 and the distal end 208 of the elongated member is configured and arranged for percutaneous insertion into a patient. In at least some embodiments, the catheter 102 defines at least one flush port, such as flush port 210. In at least some embodiments, the flush port 210 is defined in the hub 204. In at least some embodiments, the hub 204 is configured and arranged to couple to the control module (104 in FIG. 1). In some embodiments, the elongated member 202 and the hub 204 are formed as a unitary body. In other embodiments, the elongated member 202 and the catheter hub 204 are formed separately and subsequently assembled together.

Conventional mechanical ICE catheters, as opposed to solid state ICE catheters, are typically steered from outside the body by applying torque to the proximal end of the catheter to transmit torque to the distal tip where one or more ICE imaging elements are located. When the catheter passes through tortuous vasculature, the torque response of the catheter distal tip to a torque applied at the catheter proximal end may be unpredictable, potentially causing one or more undesired imaging artifacts. Accordingly, it may be advantageous to provide torque at the distal end of the catheter, thereby reducing uncertainty associated with applying torque at the proximal end of the catheter.

In the case of solid state ICE catheters, although the catheters do not need to transmit torque, the catheters typically deploy imaging elements along a length of the catheter, thereby generating images along a longitudinal axis of the catheter. Such images, in contrast to a 360-degree cross-sectional view, may not include landmarks to aid in navigation within the chambers. Accordingly, it may be advantageous to be able to provide information related to the position and orientation of the catheter when generating images.

FIG. 3 is a schematic perspective view of one embodiment of the distal end 208 of the elongated member 202 of the catheter 102. The elongated member 202 includes a sheath 302 and a lumen 304 extending along a longitudinal axis 305 of the sheath 302. An imaging core 306 is disposed in the lumen 304. The imaging core 306 is coupled to a distal end of a flexible imaging core shaft 308. The imaging core 306 includes an imaging device 310 and at least a portion of a rotational, magnetic motor 312. In at least some embodiments, the imaging device 310 includes one or more ultrasound transducers. In preferred embodiments, the imaging device 310 is fixedly coupled to the distal end of the imaging core shaft 308 such that the imaging device 310 does not rotate relative to the imaging core shaft 308. In at least some embodiments, one or more conductors 318 electrically couple the imaging device 310 to the control module 104 (See FIG. 1). In at least some embodiments, the one or more conductors 318 extend along the imaging core shaft 308. In at least some embodiments, the one or more conductors 318 extend within the imaging core shaft 308.

The motor 312 includes a magnet 314 driven to rotate by a generated magnetic field. In at least some embodiments, the magnetic field is generated by one or more magnetic-field windings (“windings”) 316. In at least some embodiments, the windings 316 are powered by one or more current lines 320 which extend to the proximal end 206 of the catheter 102. In at least some embodiments, one or more current lines 320 electrically couple the windings 316 to the control module 104 (See FIG. 1). In at least some embodiments, the one or more current lines 320 extend along the imaging core shaft 308. In at least some embodiments, the one or more current lines 320 extend within the imaging core shaft 308. In at least some alternate embodiments, an external magnetic field can be used in addition to, or in lieu of, the windings 316 to rotate the magnet 314.

In at least some embodiments, the imaging device 310 faces approximately perpendicular to a longitudinal axis of the imaging core 306 (i.e., the imaging device is side-facing). In which case, in at least some embodiments the rotation of the magnet 314 causes a corresponding rotation of the imaging device 310. In preferred embodiments, the imaging device 310 faces approximately parallel to the longitudinal axis of the imaging core 306 (i.e., the imaging device is forward-facing or rearward-facing). In which case, and as shown in FIG. 3, rotation of the magnet 314 causes a corresponding rotation of a canted (e.g., tilted) mirror 320 with a reflective surface 322 to which the imaging device 310 is directed, and from which acoustic signals are reflected. In at least some embodiments, when rotation of the magnet 314 causes rotation of the mirror 320, a mirror holder 324 may be used to transfer the rotation of the magnet 314 to the mirror 320, and also to at least partially retain the mirror 320. In at least some embodiments, a matching layer 326 is disposed between the imaging device 310 and the mirror 320.

Turning briefly to FIG. 4, the mirror holder 324 can be formed from any suitable rigid material with an aperture, or window 402, through which acoustic signals may be transmitted. The mirror holder 324 is configured and arranged to receive the mirror 320. In at least some embodiments, the mirror holder 324 is configured and arranged to at least partially receive the imaging device 310. In at least some embodiments, the mirror holder 324 is configured and arranged to receive the imaging device 310 such that rotation of the mirror holder 324 does not cause a corresponding rotation of the imaging device 130. In at least some embodiments, the mirror holder 324 is configured and arranged to receive a distal portion of the imaging core shaft 308. In at least some embodiments, the mirror holder 324 is configured and arranged to receive the matching layer 326.

Turning back to FIG. 3, the sheath 302 may be formed from any flexible, biocompatible material suitable for insertion into a patient. Examples of suitable materials include, for example, polyethylene, polyurethane, polytetrafluoroethylene (“PTFE”), other plastics, and the like or combinations thereof.

In at least some embodiments, the imaging device 310 can be used to form a radial cross-sectional image of a surrounding space. Thus, for example, when the imaging device 310 is disposed in the catheter 102 and inserted into a blood vessel of the patient, or a chamber of the patient's heart, the imaging device 310 may be used to form an image of the walls of the blood vessel or the chamber of the heart, as well as tissue surrounding the blood vessel or heart chamber.

As the imaging device 310 emits acoustic signals and receives echo signals from patient tissue, a plurality of images are formed that collectively generate a radial cross-sectional image of a portion of the region surrounding the imaging device 310, such as the walls of a blood vessel or heart chamber of interest, and the tissue surrounding the blood vessel or heart chamber of interest. In at least some embodiments, the radial cross-sectional image can be displayed on the one or more displays 112.

In at least some embodiments, the imaging core 306 may also move longitudinally along the blood vessel or heart chamber so that a plurality of cross-sectional images may be formed along an area of the blood vessel or heart chamber. In at least some embodiments, during an imaging procedure the imaging device 310 may be retracted (i.e., pulled back) along a longitudinal length of the catheter 102. In at least some embodiments, the drive unit 110 drives the pullback of the imaging core 306 within the catheter 102.

The quality of an image produced at different depths from the imaging device 310 may be affected by one or more factors including, for example, bandwidth, transducer focus, beam pattern, as well as the frequency of the acoustic pulse. The frequency of the acoustic pulse output from the imaging device 310 may also affect the penetration depth of the acoustic pulse output from the imaging device 310. In general, as the frequency of an acoustic pulse is lowered, the depth of the penetration of the acoustic pulse within patient tissue increases. In at least some embodiments, the imaging device 310 operates within a frequency range of 20 MHz to 60 MHz.

In at least some embodiments, the catheter 102 can be inserted percutaneously into a patient via an accessible blood vessel, such as the femoral artery, femoral vein, or jugular vein, at a site remote from the selected portion of the selected region, such as a blood vessel, to be imaged. The catheter 102 may then be advanced through the blood vessels of the patient to the selected imaging site, such as a portion of a selected blood vessel or a chamber of the patient's heart.

Examples of IVUS or ICE imaging systems that include imaging cores coupled to elongated members, and magnetic motors that rotate either imaging devices disposed in the imaging cores, or mirrors disposed in the imaging cores and in proximity to imaging devices, are found in, for example, U.S. application Ser. Nos. 12/415,724; 12/415,768; 12/415,791; 12/565,632; 12/566,390; 61/286,674; and 61/288,719, all of which are incorporated herein by reference.

As discussed above, the rotatable magnet 314 is disposed in the imaging core 306. The magnetic field used for driving rotation of the magnet 314 can be provided by any suitable source. In at least some embodiments, the magnetic field is generated by the windings 316. In at least some embodiments, the windings 316 are disposed in the imaging core 306 during an imaging procedure. In at least some alternate embodiments, the windings 316 are disposed external to the sheath 302 during an imaging procedure. In at least some embodiments, the windings 316 are disposed external to a patient during an imaging procedure.

The magnet 314 has a longitudinal axis about which the magnet 314 rotates. In at least some embodiments, the longitudinal axis of the magnet 314 is parallel with a longitudinal axis of the imaging core 306. In order for the magnet 314 to rotate about the longitudinal axis, the torque is applied about the longitudinal axis. Therefore, the magnetic field generated by the windings 316 lies in a plane perpendicular to the longitudinal axis of the imaging core 306, as discussed in more detail below.

The magnet 314 may be formed from many different magnetic materials suitable for implantation including, for example, neodymium-iron-boron, or the like. One example of a suitable neodymium-iron-boron magnet is available through Hitachi Metals America Ltd, San Jose, Calif. In at least some embodiments, the magnet 314 is cylindrical. In at least some embodiments, the magnet 314 is spherical. In at least some embodiments, the magnet 314 is radially symmetric, having an outside radius that varies along the length of the magnet. In at least some embodiments, the magnet 314 has a magnetization M of no less than 1.4 T, 1.5 T, 1.6 T, or more. In at least some embodiments, the magnet 314 has a magnetization vector that is perpendicular to the longitudinal axis of the magnet 314.

In at least some embodiments, the windings 316 provide a constant torque to rotate the magnet 314 at a constant frequency. In at least some embodiments, the magnet 314 rotates at a frequency of at least 1 Hz, 5 Hz, 10 Hz, 20 Hz, 30 Hz, 50 Hz, 100 Hz, 500 Hz, 1000 Hz, 1500 Hz, 2000 Hz, 2500 Hz, 3000 Hz, or higher.

It will be understood that there are many different multiple-phase winding geometries and current configurations that may be employed to form a rotating magnetic field. For example, the motor 312 may include, for example, a two-phase winding, a three-phase winding, a four-phase winding, a five-phase winding, or more multiple-phase winding geometries. It will be understood that the motor 312 may include many other multiple-phase winding geometries. In a two-phase winding geometry, the currents in the two windings are out of phase by 90°. For a three-phase winding, there are three lines of sinusoidal current that are out of phase by zero, 120°, and 240°, with three current lines also spaced by 120°, resulting in a uniformly rotating magnetic field that can drive the magnet 314 perpendicular to the current lines.

Typically, the generated magnetic field is uniform. In at least some embodiments, however, the generated magnetic field is not uniform. For example, in at least some embodiments a single winding 316 may be employed to rotate the magnet 314. In at least some embodiments, a single wire is disposed adjacent one side of the magnet 314, with a return lead disposed away from the magnet 314.

It may be an advantage to be able to image patient tissue from a variety of different angles. It may especially be an advantage when imaging in a chamber of a heart because the chambers are relatively large in size in relation to the imaging core 306. As herein described, the imaging core 306 of the imaging system is configured and arranged to at least partially extend outwards from the distal end of the sheath 302. Once the imaging core 306 is at least partially extended from the sheath 302, the imaging core shaft 308 is configured and arranged to bend to a predetermined axial angle (as shown by dotted arrows 328) (in relation to the longitudinal axis 305 of the sheath 302), thereby altering the angle of the imaging core 306 with respect to the sheath 302, such that the longitudinal axis of the imaging core 306 is not parallel with the longitudinal axis 305 of the sheath 302.

As described above, in at least some embodiments, the motor 312 drives rotation of the magnet 314 disposed in the imaging core 306 which, in turn, rotates the mirror 320. The rotating mirror 320 enables the fixed imaging device 310 to form images around the periphery of the imaging core 306. In at least some embodiments, the imaging system described herein employs an additional magnetic motor (“an imaging core shaft rotator”) configured and arranged to selectively rotate at least the distal end of the imaging core shaft 308 such that the extended (and potentially axially bent) imaging core 306 can be steered radially (as shown by dotted arrow 330), tracing a locus more or less perpendicular and about the longitudinal axis 305 of the sheath 302 to any particular radial angle. In at least some embodiments, the distal end 208 of the sheath 302 can be bent to further adjust the axial angle of the extended imaging core 306 relative to the longitudinal axis 305 of the sheath 302.

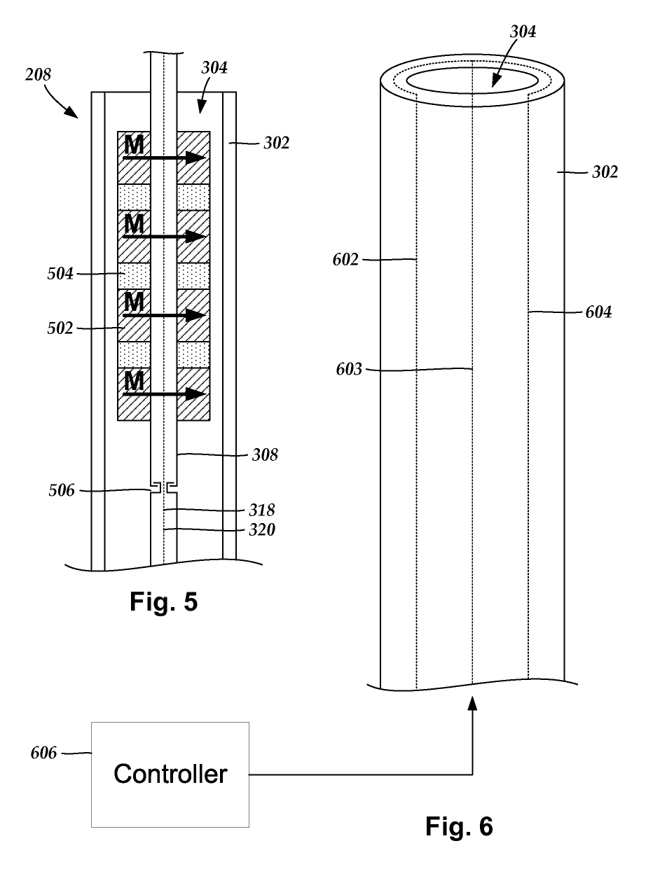

In at least some embodiments, the imaging system includes a position and orientation system for determining the position and orientation of the magnet 314 within the imaging core 306. In at least some embodiments, the position and orientation system can determine the orientation of the imaging core shaft magnets (502 in FIG. 5) within the sheath 302.

In at least some embodiments, the imaging core shaft rotator includes a plurality of rotatable magnets coupled to the imaging core shaft 308 (“imaging core shaft magnets”). The imaging core shaft magnets are driven to rotate by a magnetic field generated at the location of the imaging core shaft magnets. In at least some embodiments, the magnetic field is generated by one or more windings. In at least some embodiments, the one or more windings used to generate the magnetic field at the location of the one or more rotatable imaging core shaft magnets are disposed in the sheath. In at least some alternate embodiments, the one or more windings used to generate the magnetic field at the location of the one or more rotatable imaging core shaft magnets are external to the patient.

In at least some embodiments, the distal end 208 of the sheath 302 is open and the imaging core 306 is configured and arranged for extending outwardly from the distal end 208 of the sheath 302. FIG. 5 is a schematic longitudinal cross-sectional view of one embodiment of a portion of the imaging core shaft 308 disposed in the distal end 208 of the sheath 302. In at least some embodiments, a plurality of imaging core shaft magnets, such as imaging core shaft magnet 502, are coupled to the imaging core shaft 308. In at least some embodiments, the imaging core shaft magnets 502 at least partially encircle the imaging core shaft 308. In at least some embodiments, one or more flexible spacers, such as flexible spacer 504, are disposed between adjacent imaging core shaft magnets 502. In at least some embodiments, the flexible spacers 504 increase the flexibility of the imaging core shaft 308 in proximity to the imaging core shaft magnets 502.

In at least some embodiments, the imaging core shaft 308 includes a linkage 506. In at least some embodiments, the portion of the imaging core shaft 308 distal to the linkage 506 is rotatable, while the portion of the imaging core shaft 308 proximal to the linkage 506 is non-rotatable. In at least some embodiments, the imaging core shaft magnets 502 are coupled to the imaging core shaft 308 such that the imaging core shaft magnets 502 are distal to the linkage 506. In at least some embodiments, the imaging core shaft magnets 502 are coupled to the imaging core shaft 308 such that the imaging core shaft magnets 502 are proximal to the portion of the imaging core shaft 308 that extends from the distal end 208 of the sheath 302.

Rotation of the imaging core shaft magnets 502 causes a corresponding radial rotation of the imaging core shaft 308 distal to the linkage 506. In at least some embodiments, the radial rotation of the imaging core shaft 308 causes a corresponding radial rotation of the imaging core 306 around the longitudinal axis 305 of the sheath 302.

The imaging core shaft magnets 502 are driven to rotate by a magnetic field generated at the location of the imaging core shaft magnets 502. In at least some embodiments, the magnetic field is generated by one or more windings (e.g., windings 602-604 of FIG. 6). In at least some embodiments, the windings are adjacent to the imaging core shaft magnets 502. In alternate embodiments, the windings are disposed external to the sheath 302. In at least some embodiments, the windings are disposed external to a patient during an imaging procedure. In at least some embodiments, controlling the current into the windings controls the angular position of the imaging core shaft 308 (i.e., the direction of magnetization vector M in the plane perpendicular to the imaging core shaft 308) which, in turn controls the orientation of the imaging core 306 around the longitudinal axis of the sheath 302.

FIG. 6 is a schematic perspective view of one embodiment of windings embedded in the sheath 302. Any suitable number of windings may used. In at least some embodiments, the sheath 302 includes a three-phase winding 602-604 that provides the magnetic field to rotate the imaging core shaft 308 such that the extended imaging core 306 rotates radially (i.e., is steered) around the sheath 302. In at least some alternate embodiments, an external magnetic field can be used in addition to, or in lieu of, the three-phase winding 602-604 to rotate the imaging core shaft 308 around the sheath 302.

In at least some embodiments, a controller 606 is coupled to the windings 602-604. In at least some embodiments, the controller 606 is external to the patient during an imaging procedure. In at least some embodiments, the controller 606 includes an electronic subsystem for controlling one or more operations of the imaging device 100, such as drive electronics and controls, transmit and receive electronics, and image processing and display electronics. In at least some embodiments, the controller 606 includes a dial for adjusting current input to the windings 602-604. In at least some embodiments, adjusting the current input to the windings 602-604 controls the steering of the imaging core shaft 308. In at least some embodiments, the controller 606 includes a power supply, such as one or more batteries. In at least some embodiments, the controller 606 is coupled to the control module 104.

In at least some embodiments, multiple windings may occupy a single layer on a cylindrical surface with no cross-overs. In FIG. 6, windings 602-604 are shown as being single-layer windings. In at least some embodiments, the windings 602-604 are free standing metal strips cut from the surface of a metal cylinder. In other embodiments, single layer windings or strips may be deposited on a non-conductive cylindrical surface. Such a winding may occupy a minimal volume in an insertable medical device. Although other geometries may also form a rotating magnetic field, the three-phase geometry may have the advantages of allowing for a more compact motor construction than other geometries that require multiple turns with cross-overs that add to the radial dimension of the motor.

One useful property of a three-phase winding is that only two of the three windings 602-604 needs to be driven, while the third line is a common return that mathematically is equal to the third phase of current. This can be verified by noting that:

Sin(Ψ)+Sin(Ψ+120°)=−Sin(Ψ+240°)

For a three-phase winding, current is driven into two lines with the zero and 120° phase shift of the two terms on the left side of this identity. The sum of the two terms returns on the common line with exactly the correct 240° phase shift on the right side of this equation needed to create a magnetic field directed at angle Ψ relative to the line with zero phase shift. It will be understood that the minus sign indicates that the return current is in the opposite direction of driven current. The magnetization M of the imaging core shaft magnets 502 will align with the magnetic field at angle Ψ, minus a lag angle that is determined by the overall drag or friction in imaging core shaft 308. The angle w may be selected by the system operator to provide a desired viewing angle. The processor (106 in FIG. 1) may generate currents in windings 602-604 that result in a sequence of imaging angles Ψ.

In at least some embodiments, the three unsupported windings 602-604 may be supported by a substrate to increase mechanical stability. In at least some embodiments, the windings 602-604 are constructed from a solid metal tube, leaving most of the metal intact, and removing only metal needed to prevent shorting of the windings 602-604. In at least some embodiments, the removed portions are backfilled with a non-conductive material. In at least some embodiments, the windings 602-604 each have an overall wall thickness of no greater than 60 μm, 50 μm, or 40 μm.

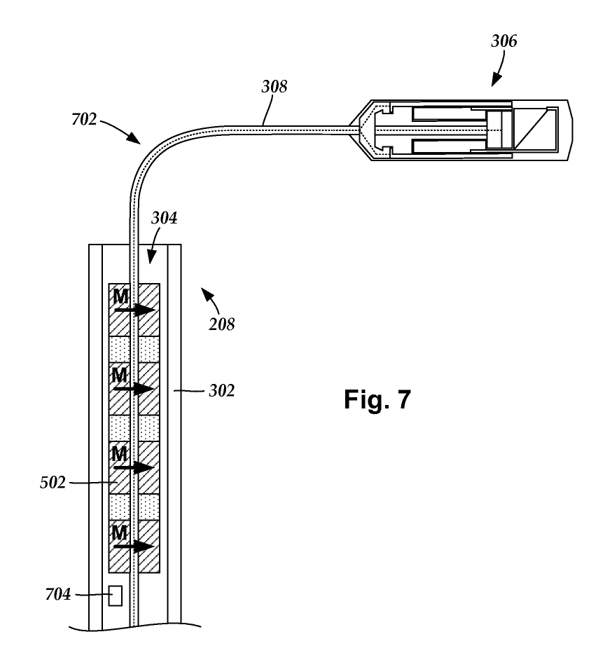

FIG. 7 is a schematic view of one embodiment of the imaging core 306 extended from the distal end 208 of the sheath 302. The imaging core 306 is tethered to the sheath 302 by the imaging core shaft 308, which extends along the lumen 304. The imaging core shaft 308 includes a region 702 that forms a predetermined axial bend when the region 702 is at least partially extended from the sheath 302. In at least some embodiments, the bent region 702 is formed from one or more shape memory materials. In at least some embodiments, the bent region 702 has a predetermined axial bend of at least 5°, 10°, 15°, 20°, 25°, 30°, 35°, 40°, 45°, 50°, 55°, 60°, 65°, 70°, 75°, 80°, 85°, 90°, 95°, 100°, 105°, 110°, 115°, 120°, 125°, 130°, 135°, 140°, 145°, 150°, 155°, 160°, 165°, 170°, 175°, 180°, or more relative to the sheath 302. In FIG. 7, the predetermined angle of the bent region 702 is shown as being approximately 90° relative to the sheath 302. It will be understood that the bent region 702 can include multiple bends. Also, the imaging core shaft 308 can include more than one bent region 702.

In at least some embodiments, the imaging system includes one or more position and orientation systems. In at least some embodiments, a position and orientation system is configured and arranged to detect the position and orientation of a static magnetic field or of a magnetic field that is rotating at a frequency of no less than 1 Hz, 5 Hz, 10 Hz, 20 Hz, 30 Hz, 50 Hz, 100 Hz, 500 Hz, 1000 Hz, 1500 Hz, 2000 Hz, 2500 Hz, or 3000 Hz. In at least some embodiments, a position and orientation system is configured and arranged to detect the position and orientation of the magnet 314. In at least some embodiments, a position and orientation system is configured and arranged to detect the position and orientation of the imaging core shaft magnets 502.

In at least some embodiments, the position and orientation system includes an array of magnetic sensors positioned outside the patient that synchronously detects the magnetic field created by the magnet 314 as the magnet 314 rotates. In at least some embodiments, the currents driving the rotating magnet 314 may be used as a reference in a synchronous detection system to provide high resolution measurements. There are many ways to sense a magnetic field. A coil of wire can sense AC magnetic fields. The sensitivity, or signal to noise ratio, of the induction coil increases with the coil volume. Thus, large coils can be more sensitive than relatively smaller coils. If compact, small-volume sensors are desired for a given application, then modern sensors, such as giant magnetoresistance (“GMR”) sensors, may increase sensitivity.

The magnetic gradient tensor is measured and inverted using a known algorithm to produce the Cartesian coordinates and orientation of the rotating magnet 314. Without wishing to be held to any particular values, calculations using commercially available magnetic field sensors show that a location of the magnet 314 may be localized to sub-millimeter accuracy when the rotating magnet 314 has an 0.8 mm diameter and a 5 mm length and an array of magnetic sensors is located up to 0.5 meters from the rotating magnet 314. The accuracy may be improved using many different techniques including, for example, increasing the size of the rotating magnet 314, increasing the saturation magnetization of the magnet material, increasing the speed of rotation of the magnet 314, increasing the interval over which data are averaged (i.e., reducing the sampling rate), increasing the volume of the sensors, increasing the sensitivity of the sensors, reducing the distance between the rotating magnet 314 and the sensor array (114 in FIG. 1), increasing the number of magnetic sensors, improving the relative locations of the sensors in the sensor array (114 in FIG. 1), sensing the angular position of the magnet 314 as it rotates and providing this data as a reference for a lock in amplifier whose input is a magnetic field sensor, or the like or combinations thereof.

In at least some embodiments, the sensor array 114 includes an array of magnetic field sensors that are external to the patient. The sensor array 114 may include like magnetic field sensors chosen for a particular application. In addition to GMR sensors, other suitable magnetic sensors include, for example, magnetic induction (wire wound around a magnetic core), flux gate magnetometers, saturable core magnetometers, Hall Effect, Superconducting Quantum Interference Device (“SQUID”) magnetometers, or the like. In at least some embodiments, the array of magnetic sensors 114 are positioned within a block.

In at least some embodiments, the position and orientation system additionally includes a computer (e.g., the processor 106 of FIG. 1) that computes the position and orientation of the rotating magnet 314 from the sensed magnetic field data. In at least some embodiments, the position and orientation system synchronously detects a specific rate of rotation of the rotating magnet 314. In at least some embodiments, the output of a miniature sensor that detects the angular position of the rotating magnet 314 may be used as a reference for a lock-in amplifier that measures the sensed magnetic field of the rotating magnet 314. In at least some embodiments, one or more currents injected into the windings 316 may be used as a reference for the lock-in amplifier.

In at least some embodiments, the position and orientation system can be used to detect the orientation of the imaging core shaft magnets 502. In at least some embodiments, the position and orientation system includes a DC magnetic sensor 704 disposed on, or in proximity to, one or more of the imaging core shaft magnets 502 disposed on the imaging core shaft 308.

In at least some embodiments, when the imaging core 306 is extended from the sheath 302, the distal end 208 of the sheath 302 can also be deflected axially. Thus, when the imaging core 306 is extended from the sheath 302 and the distal end of the imaging core shaft 308 is bent to a predetermined axial angle relative to the sheath 302, deflecting the distal end 208 of the sheath 302 can provide a way to further adjust the axial angle of the imaging core 306 relative to the sheath 302.

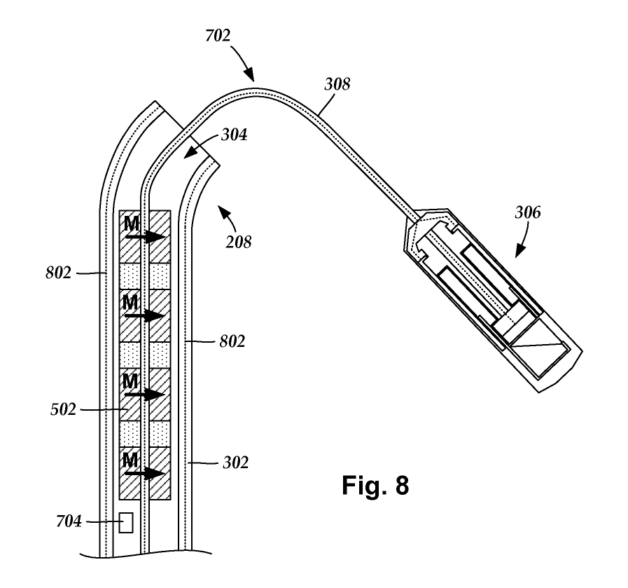

FIG. 8 is a schematic view of one embodiment of the imaging core 306 extended from the distal end 208 of the sheath 302. The distal end 208 of the sheath 302 is deflected, thereby adjusting the axial angle of the imaging core 306 relative to the sheath 302. The distal end 208 of the sheath 302 can be deflected using any suitable mechanism. In at least some embodiments, one or more pull wires 802 are disposed in the sheath 302. In at least some embodiments, the one or more pull wires 802 are coupled to the sheath 302 in proximity to the distal end 208 and extend to the proximal end. In at least some embodiments, the pull wires 802 (or one or more distal portions of the pull wires 802) may be formed as shape memory (e.g., Nitinol) wires that are heated via currents supplied by leads to transition between a low temperature state and a high temperature state. In at least some embodiments, the shape memory pull wires 802 may be thermally insulated to avoid heating their surroundings.

As shown in FIGS. 5, 7, and 8, the imaging core shaft magnets 502 are all magnetized in the same direction (shown as arrows positioned over the imaging core shaft magnets 502), perpendicular to the longitudinal axis of the imaging core shaft 308. In at least some embodiments, torque is applied to the imaging core shaft magnets 502 from the three lines of current 602-604 flowing in the wall of the sheath 302. The current lines 602-604 form a three-phase winding, with driven currents I₁ and I₂ returning in the third line. If the relative magnitudes of the two driven currents are properly selected, a magnetic field is produced by the three current lines 602-604 that is perpendicular to the longitudinal axis of the imaging core shaft 308. The torque on a given magnet of the imaging core shaft magnets 502 is equal to the cross product of the magnet's magnetic moment and the magnetic field created by the winding currents. The torque is about the drive shaft axis, and is proportional to the number of shaft magnets.

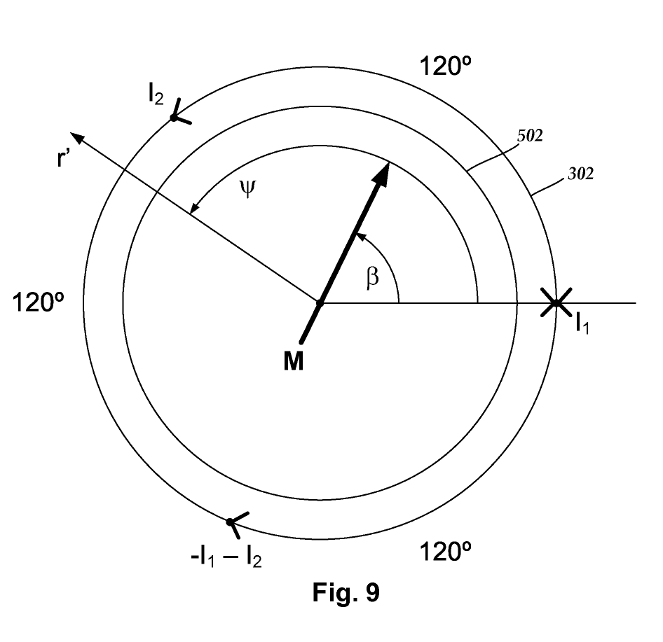

FIG. 9 is a schematic transverse cross-sectional view of one embodiment of one of the imaging core shaft magnets 502 disposed in the sheath 302. If the currents in the three phase lines 602-604 are given by:

I₁=I₀ sin(Ψ);

I₂=I₀ sin(Ψ+120°); and

I₃=−I₁−I₂=I₀ sin(Ψ+240°); (1)

then the magnetic field at the center of the imaging core shaft 308 is given by:

H=(3I₀/2πD)r′; (2)

where
H=vector magnetic field in Amps/m;
Iₒ=current amplitude defined by Eq. (1) in amps;
D=outer sheath diameter in meters; and
r′=unit radius vector at angle Ψ, defined in FIG. 3.
   If the magnet magnetization vector is oriented at angle φ, as shown in FIG. 9, then the torque generated by the magnetic field of Eq. (2) is around the longitudinal axis of the imaging core shaft 308, and has magnitude:

τ=(3/8D)MNL(d₂²−d₁²)I₀ sin(Ψ−φ); (3)

where
τ=imaging core shaft torque in Nt-m;
M=magnet magnetization in Tesla;
L=individual magnet length in meters;
N=number of magnets;
d₂=magnet outside diameter in meters; and
d₁=magnet inside diameter in meters.

The generated torque of the imaging core shaft 308 is proportional to the number of imaging core shaft magnets 502, and the length and cross sectional area of the individual imaging core shaft magnets 502 and the current in the windings 602-604. As the imaging core shaft magnet 502 magnetization M aligns with the magnetic field vector, the angle Ψ−φ tends toward zero. For small angles, when the imaging core shaft magnets 502 are nearly aligned with the magnetic field, the torque is proportional to the difference angle, providing a linear restoring torque for angular displacements of the imaging core shaft magnets 502 away from the field direction.

For open loop operation, with current applied, as the imaging core shaft magnets 502 turn, they resist a restoring torque τᵣ provided by frictional contact between the imaging core shaft magnets 502 and an inner surface of the sheath 302 and wind up in the imaging core cable 318. In at least some embodiments, imaging core shaft wind up torque can be reduced by providing the rotation linkage (506 in FIG. 5). Setting the applied torque in Eq. (3) equal to the restoring torque gives an expression for the deviation angle:

Ψ−φ=sin−1(τᵣ/[3/8D)MNL(d₂²−d₁²)I₀]). (4)

The deviation angle will tend to zero as the number of imaging core shaft magnets 502 or the drive current is increased, or the restoring torque is reduced. In the imaging system 100, the totality of angular positions that needs to be addressed is −180°<φ<180°, so friction and wind up restoring torques may be small, and the imaging core shaft magnet angle may be close to the selected field angle over this range. The current may be set to a practical value that is just below a level that generates heat in the sheath 302. If open loop operation results in a sufficiently small deviation angle, then the prescription for obtaining a given angle φ˜Ψ is given by Eq. (1), where I₀ is a maximum practical operating current. Optionally, cooling fluid can be injected into the sheath 302 to reduce the temperature of the windings 602-604, thereby allowing more current to flow in the windings 602-604 and, consequently, providing more torque to the imaging core shaft magnets 502.

For closed loop operation, the localization sensor system, or the DC magnet orientation sensor 704, monitors the positions of the imaging core shaft magnets and feeds the information back to the magnetic field orientation angle, Ψ, increasing the deviation angle and torque in Eq. (3) until the measured imaging core shaft magnet angle is equal to the desired angle. This assumes that the current is set to the practical value just below a level that generates heat in the sheath 302 (if not, feedback could optionally, or additionally, be used to increase current). Since the sine function has a peak value of unity, angle feedback can only be used to increase torque in Eq. (3) to an upper value of:

τₘₐₓ˜(3/8D)MNL(d₂²−d₁²)I₀; (5)

which occurs when Ψ=φ+90°. The prescription for the applied currents in closed loop operation is still given by Eq. (1).

Optionally, for open loop operation, current applied to the windings 602-604 can be used, for example, to scan the imaging core through a variety of angles. Additionally, the orientation of the imaging core 306 can be sensed as it is moving and imaging, using the external sensor array 114. Images can be displayed at the measured angles.

To estimate the magnitude of the torque, consider an exemplary sheath 302 with a diameter D=2.5 mm, a total imaging core shaft magnet length of NL=10 cm, with individual imaging core shaft magnets 502 having an outside diameter of 2 mm and inside diameter of 0.5 mm. The current is taken as 6 amps through 0.5 mm diameter leads (AWG #24), producing about 1 Watt of heat per foot of sheath length, which should be dissipated without a significant temperature rise. Note that the windings 602-604 can consist of multiple turns of finer wire, each carrying less current (e.g., ten turns @ 0.6 amps) to give the same torque and same Ohmic heat. The torque computed from Eq. (5) is then about 500 μN-m or 50 gm-mm. At the 1 mm radius of the sheath 302, this is a force of 50 grams (or a few ounces), which may be more than adequate to overcome friction with the sheath 302 and wind up torque in the center cable 318. Additionally, as discussed above, it may be possible to increase the amount of torque by optionally injecting cooling fluid into the sheath 302.

If the deflected distal end 208 of the sheath 302 shown in FIG. 8 has a length of 10 mm, the torque transmitted to the distal end 208 in this example would be 50 gm-mm divided by 10 mm=5 grams. This is on the order of the tissue contact force exerted by the at least one other known catheter tip.

In practice, the user may operate a dial to control current in the windings 602-604 and a pull wire lever to bend the sheath 302, thereby steering the imaging core 306 to a desired location and angle. In an automated system, the user might indicate an imaging view via point and click on a 3-D image of a heart chamber, after which a computer (e.g., the processor 106 of FIG. 1) controls the winding currents and pull wires 802. Algorithms known in the prior art can direct the distal end 208 of the sheath 302 in a critically damped fashion to the indicated location without excessive overshoot and hunting for the target location. In a sweep mode, the computer may direct a sequence of sheath bends and current sweeps to acquire an entire 3-D image of the heart chamber. This image may be taken first, and used as the road map image for subsequent navigation.

In at least some embodiments, a rotating external magnetic field may be used to rotate the imaging device 310 at the distal end 208 of the sheath 302. In at least some embodiments, orienting the external magnetic field away from the magnetization vector of the magnet 314 causes the distal end 208 of the sheath 302 to deflect, eliminating the need for pull wires 802. If the rotating external magnetic field is large enough, the external rotating magnetic field could provide both deflection torque and rotational torque, thereby potentially eliminating the need for the imaging core shaft magnets 502 and sheath stator windings 602-604, as well.

Increasing the number of imaging core shaft magnets 502 on the imaging core shaft 308 may increase the amount of torque generated. In at least some embodiments, generated torque may be used, for example, to provide a measured tissue contact force during an ablation procedure, such as electrophysiology ablation.

The above specification, examples and data provide a description of the manufacture and use of the composition of the invention. Since many embodiments of the invention can be made without departing from the spirit and scope of the invention, the invention also resides in the claims hereinafter appended.

1. A medical imaging assembly comprising:
an elongated sheath having a proximal end, an open distal end, and a longitudinal axis, wherein the sheath defines a lumen that extends to the open distal end;
an imaging core shaft at least partially disposed in the lumen, the imaging core shaft having a proximal end, a distal end, and a longitudinal axis, wherein the distal end of the imaging core shaft comprises a shape memory region;
a sealed imaging core disposed at the distal end of the imaging core shaft, wherein the imaging core is configured and arranged for extending outward from the open distal end of the sheath, wherein when the shape memory region of the imaging core shaft is at least partially extended from the distal end of the sheath, the shape memory region is configured and arranged to bend axially with respect to the longitudinal axis of the sheath such that the longitudinal axis of the sheath is not parallel with the longitudinal axis of the imaging core, the imaging core comprising
an imaging core magnet disposed at a location in the imaging core, the imaging core magnet configured and arranged to rotate at a target frequency by generation of a magnetic field at the location of the imaging core magnet,
at least one transducer configured and arranged for transforming applied electrical signals to acoustic signals, transmitting the acoustic signals, receiving corresponding echo signals, and transforming the received echo signals to electrical signals, and
a mirror comprising a reflective surface, the mirror configured and arranged for reflecting acoustic signals transmitted from the at least one transducer and corresponding echo signals, wherein the mirror is coupled to the imaging core magnet such that rotation of the imaging core magnet causes a corresponding rotation of the mirror;
an imaging core shaft rotator configured and arranged to rotate the imaging core shaft such that, when the imaging core is at least partially extended from the open distal end of the sheath, rotation of the imaging core shaft causes a corresponding radial rotation of the imaging core about the longitudinal axis of the sheath, the imaging core shaft rotator comprising a plurality of rotatable imaging core shaft magnets fixedly disposed over a portion of the imaging core shaft; and
at least one transducer conductor electrically coupled to the at least one transducer and in electrical communication with the proximal end of the catheter.

2. The medical imaging assembly of claim 1, further comprising a mirror holder coupling the imaging core magnet to the mirror.

3. The medical imaging assembly of claim 2, wherein the at least one transducer is at least partially disposed within the mirror holder.

4. The medical imaging assembly of claim 1, further comprising at least one first magnetic-field winding, the at least one first magnetic-field winding configured and arranged to generate the magnetic field at the location of the imaging core magnet.

5. The medical imaging assembly of claim 4, wherein the at least one first magnetic-field winding is disposed in the imaging core.

6. The medical imaging assembly of claim 4, further comprising at least one current line coupled to the at least one first magnetic-field winding and in electrical communication with the proximal end of the sheath.

7. The medical imaging assembly of claim 6, wherein the at least one current line extends within at least a portion of the imaging core shaft.

8. The medical imaging assembly of claim 1, further comprising at least one second magnetic-field winding, the at least one second magnetic-field winding configured and arranged to generate the magnetic field at the location of the plurality of imaging core shaft magnets.

9. The medical imaging assembly of claim 8, wherein the at least one second magnetic-field winding is at least partially embedded in the sheath.

10. The medical imaging assembly of claim 8, further comprising a controller coupled to the at least one second magnetic-field winding, the controller configured and arranged to adjust an amount of current applied to the at least one second magnetic-field winding.

11. The medical imaging assembly of claim 1, wherein the plurality of imaging core shaft magnets are disposed over a portion of the imaging core shaft that remains within the lumen of the sheath when the imaging core is extended from the open distal end of the sheath.

12. The medical imaging assembly of claim 1, further comprising at least one pull wire coupled to the distal end of the sheath and extending to the proximal end of the sheath.

13. The medical imaging assembly of claim 12, wherein the at least one pull wire is formed from a shape memory material configured and arranged to change shape upon exposure to at least one of heat or electric current.

14. The medical imaging assembly of claim 1, further comprising at least one flexible spacer disposed between two adjacent imaging core shaft magnets of the plurality of imaging core shaft magnets.

15. A medical imaging system comprising:
the medical imaging assembly of claim 1; and
a control module coupled to the imaging core, the control module comprising
a pulse generator configured and arranged for providing electric signals to the at least one transducer, the pulse generator electrically coupled to the at least one transducer via the at least one transducer conductor, and
a processor configured and arranged for processing received electrical signals from the at least one transducer to form at least one image, the processor electrically coupled to the at least one transducer via the at least one transducer conductor.

16. The medical imaging system of claim 15, further comprising a position and orientation system configured and arranged for determining the position and orientation of the imaging core magnet.

17. The medical imaging system of claim 16, wherein the position and orientation system comprises an array of magnetic field sensors disposed external to a patient, the magnetic field sensors configured and arranged to sense the location and orientation of the imaging core magnet in relation to the array of magnetic field sensors.

18. The medical imaging system of claim 17, wherein the array of magnetic field sensors are coupled to the processor.

19. The medical imaging system of claim 15, further comprising a DC magnetic sensor disposed on, or in proximity to, the plurality of imaging core shaft magnets disposed on the imaging core shaft.

20. The medical imaging system of claim 19, further comprising a position and orientation system configured and arranged for determining the position and orientation of the DC magnetic sensor in relation to the array of magnetic field sensors.
